(19) United States
(12) Patent Application Publication
(10) Pub. No.: US 2025/0057641 A1
(43) Pub. Date: Feb. 20, 2025

(54) Vascular Implant

(71) Applicant: BARD PERIPHERAL VASCULAR, INC., Franklin Lakes, NJ (US)

(72) Inventors: Matt Casiraro, Tempe, AZ (US); Alexander Lastovich, Gilbert, AZ (US)

(73) Assignee: BARD PERIPHERAL VASCULAR, INC., Franklin Lakes, NJ (US)

(21) Appl. No.: 18/724,234

(22) PCT Filed: Dec. 30, 2021
(86) PCT No.: PCT/US2021/073175

§ 371 Date: Jun. 26, 2024

(51) Int. Cl. A61F 2/01

(52) U.S. Cl. CPC A61F 2/0105; A61F 2002/016; A61F 2210/0004; A61F 2220/0016; A61F 2250/0036; A61F 2250/0071

(57) ABSTRACT

A vascular implant. comprising a polymeric filter body having a central longitudinal axis and configured to be placed in a patient's blood conveying vessel portion. The filter body has a head and an outer periphery. Multiple filter legs extend both radially and longitudinally from said head. Each leg tapes between the axis and said outer periphery. Each leg has a proximal end portion next to the head and a distal portion with an anchor for engaging a vessel wall. A weakened break point is on one or more of the legs. The filter body and legs are 3D printed of a bioresorbable polymer.

### CROSS-REFERENCE TO RELATED APPLICATIONS

N/A

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

### 1. Field of the Invention

The present invention relates to bioresorbable vascular implants such as filters (e.g., vena cava filters) and occlusion devices. More particularly, the present invention relates to an improved vascular implant that bioresorbs into a patient's vascular system (e.g., inferior vena cava, iliofemoral vein, ovarian vein, splenic artery, uterine artery, hepatic artery or other vein/artery vessel). For example, the present invention bioresorbs into a patient's vascular system (e.g. inferior vena cava or iliofemoral vein) after transient risk of pulmonary embolism (PE) has subsided. The entire implant structure could be made of bioresorbable material so that no implant or implant remnant/element would ultimately be left behind as the entire implant could resorb into vascular tissue. In one or more embodiments, the implant is specially configured and 3D printable. Break points have a controlled break (or breaks) at selected locations.

### 2. General Background of the Invention

Vascular implants include various devices that are placed at a selected locale in a patient's blood vessel. One example is a vena cava filter. Another example is an occlusion device. Various patents have issued for vascular implants. Patents have also issued that relate in general to 3D printing of implants. Examples (each incorporated herein by reference) are listed in the following Table 1.

TABLE 1

| PATENT OR | | ISSUE DATE |
|---|---|---|
| PUBLICATION NO. | DESCRIPTION | (DD/MM/YYYY) |
| 8,092,484 | EMBOLUS BLOOD CLOT FILTER WITH | 10/01/2012 |
| | POST DELIVERY ACTUATION | |
| 8,092,485 | RECOVERABLE INFERIOR VENA | 10/01/2012 |
| | CAVA FILTER | |
| 8,317,818 | REMOVABLE BLOOD CLOT FILTER | 27/11/2012 |
| | WITH EDGE FOR CUTTING THROUGH | |
| | THE ENDOTHELIUM | |
| 8,420,113 | BIODEGRADABLE MEDICAL DEVICES | 16/04/2013 |
| | WITH ENHANCED MECHANICAL | |
| | STRENGTH AND PHARMACOLOGICAL | |
| | FUNCTIONS | |
| 8,518,072 | JUGULAR FEMORAL VENA CAVA | 27/08/2013 |
| | FILTER SYSTEM | |
| 8,562,638 | EMBOLUS BLOOD CLOT FILTER WITH | 22/10/2013 |
| | FLOATING FILTER BASKET | |
| 8,734,479 | EMBOLUS BLOOD CLOT FILTER | 27/05/2014 |
| | DELIVERY SYSTEM | |
| 8,777,975 | EMBOLUS BLOOD CLOT FILTER WITH | 15/07/2014 |
| | BIO-RESORBABLE COATED FILTER | |
| | MEMBERS | |
| 8,795,351 | MIGRATION RESISTANT EMBOLIC | 05/08/2014 |
| | FILTER | |
| 8,870,943 | STENT STRUCTURE FOR | 28/10/2014 |
| | IMPLANTATBLE MEDICAL DEVICE | |
| 8,992,562 | FILTER DELIVERY SYSTEM | 31/03/2015 |
| 9,220,588 | SYSTEMS, METHODS AND DEVICE | 29/12/2015 |
| | FOR EMBOLIC PROTECTION | |
| 9,393,095 | JUGULAR FEMORAL VENA CAVA | 19/07/2016 |
| | FILTER SYSTEM | |
| 9,421,081 | EMBOLUS BLOOD CLOT FILTER | 23/08/2016 |
| | DELIVERY SYSTEM | |
| 9,445,895 | INTRACARDIAC CAGE AND METHOD | 25/09/2016 |
| | OF DELIVERING SAME | |
| 9,456,888 | REVERSIBLE VASCULAR FILTER | 04/10/2016 |
| | DEVICES AND METHODS | |
| 9,468,513 | EMBOLUS BLOOD CLOT FILTER WITH | 18/10/2016 |
| | BIO-RESORBABLE COATED FILTER | |
| | MEMBERS | |
| 9,561,094 | DEVICES AND METHODS FOR | 07/02/2017 |
| | TREATING VENOUS DISEASES | |
| 9,597,435 | MEDICAL DEVICES HAVING A | 21/03/2017 |
| | BIORESORBABLE COATING LAYER | |
| | WITH A PRE-DETERMINED PATTERN | |
| | FOR FRAGMENTATION | |
| 9,693,851 | FILTER DELIVERY SYSTEM | 14/07/2017 |
| 9,730,781 | EMBOLUS BLOOD CLOT FILTER | 15/08/2017 |
| | REMOVAL SYSTEM AND METHOD | |
| 9,949,816 | IVE FILTER RETRIEVAL SYSTEMS | 24/04/2018 |
| | WITH MULTIPLE CAPTURE MODES | |
| 9,980,804 | VENA CAVA FILTER WITH FILAMENT | 29/05/2018 |
| 10,105,206 | INFERIOR VENA CAVA FILTER WITH | 23/10/2018 |
| | STABILITY FEATURES | |
| 10,188,496 | VENA CAVA FILTER FORMED FROM A | 29/01/2019 |
| | SHEET | |
| 10,188,498 | EMBOLUS BLOOD CLOT FILTER | 29/01/2019 |
| | DELIVERY SYSTEM | |
| 10,226,322 | JUGULAR FEMORAL VENA CAVA | 12/03/2019 |
| | FILTER SYSTEM | |
| 10,258,454 | VISUAL STABILIZER ON ANCHOR | 16/04/2019 |
| | LEGS OF VENA CAVA FILTER | |
| 10,279,078 | CROSSLINKABLE 3D PRINTED | 07/05/2019 |
| | BIOMATERIAL-BASED IMPLANTS | |
| | AND METHODS OF MANUFACTURE | |
| | THEREOF | |
| 10,299,906 | EMBOLUS BLOOD CLOT FILTER | 28/05/2019 |
| | UTILIZABLE WITH SINGLE DELIVERY | |
| | SYSTEM OR A SINGLE RETRIEVAL | |
| | SYSTEN IN ONE OF A FEMORAL OR | |
| | JUGULAR ACCESS | |
| 10,342,654 | IVC FILTER WITH TRANSLATING | 09/07/2019 |
| | HOOKS | |
| 10,368,972 | EMBOLUS BLOOD CLOT FILTER WITH | 06/08/2019 |
| | BIO-RESORBABLE COATED FILTER | |
| | MEMBERS | |
| 10,390,925 | MIGRATION RESISTANT EMBOLIC | 27/08/2019 |
| | FILTER | |
| 10,441,689 | METHODS AND DEVICES FOR THREE- | 15/10/2019 |
| | DIMENSIONAL PRINTING OR | |
| | ADDITIVE MANUFACTURING OF | |
| | BIOACTIVE MEDICAL DEVICES | |
| 10,470,865 | VASCULAR FILTER DEVICE | 12/11/2019 |
| 10,492,898 | EMBOLUS BLOOD CLOT FILTER AND | 03/12/2019 |
| | DELIVERY SYSTEM | |
| 10,512,531 | FILTER DELIVERY SYSTEM | 24/12/2019 |
| 10,531,942 | ABSORBABLE VASCULAR FILTER | 14/01/2020 |
| 10,579,755 | METHOD FOR 3-DAY PRINTING A | 03/03/2020 |
| | CUSTOM BONE GRAFT | |
| 10,624,731 | VASCULAR FILTER SYSTEM | 21/04/2020 |
| 10,729,527 | REMOVABLE EMBOLUS BLOOD CLOT | 04/08/2020 |
| | FILTER | |
| 10,813,738 | TUBULAR FILTER | 27/10/2020 |
| 10,842,608 | VENA CAVA FILTER WITH FILAMENT | 24/11/2020 |
| 2007/064731 | TRANSMISSION APPARATUS WITH | 22/03/2007 |
| | FUNCTION OF MULTI-STEP | |
| | BANDWIDTH ASSIGNMENT TO OTHER | |
| | COMMUNICATION APPARATUSES | |
| 2010/0074934 | MEDICAL IMPLANTS WITH A | 25/03/2010 |
| | COMBINATION OF COMPOUNDS | |
| 2016/0166371 | ENDOLUMINAL FILTER DESIGN | 16/06/2016 |
| | VARIATIONS | |
| 2016/0175085 | ENHANCED FLUOROGENIC | 23/06/2016 |
| | ENDOLUMINAL FILTER STRUCTURE | |
| 2017/0105830 | BIODEGRADABLE VASCULAR FILTER | 20/04/2017 |
| 2017/0218228 | THREE DIMENSIONAL PRINTING OF | 03/08/2017 |
| | BIO-INK COMPOSITIONS | |
| 2017/0249440 | 3D PRINTING SURGICAL REPAIR | 31/08/2017 |
| | SYSTEMS | |
| 2017/0340429 | VASCULAR FILTER SYSTEM | 30/11/2017 |
| 2018/0168811 | NOVEL BIODEGRADABLE AND NON- | 21/06/2018 |
| | BIODEGRADABLE 3D PRINTED | |
| | IMPLANTS AS A DRUG DELIVERY | |
| | SYSTEM | |
| 2018/0296343 | 3-D PRINTING OF POROUS IMPLANTS | 18/10/2018 |
| 2018/0303616 | 3-D PRINTING OF BONE GRAFTS | 25/10/2018 |
| 2018/0311028 | VENA CAVA FILTER WITH FILAMENT | 01/11/2018 |
| 2019/0110880 | MEDICAL DEVICES AND ANCHORS | 18/04/2020 |
| | THREFOR | |
| 2020/0001540 | ADDITIVE MANUFACTURING ON | 02/01/2020 |
| | UNCONSTRAINED FREEDORM | |
| | SURFACES | |
| 2020/0197150 | VASCULAR FILTER SYSTEM | 25/06/2020 |
| WO2007064731 | HELICAL VENA CAVA FILTER | 07/06/2007 |
| WO2011079287 | REVERSIBLE VASCULAR FILTER | 30/06/2011 |
| | DEVCIES AND METHODS FOR USING | |
| | SAME | |
| WO2016154148 | ARTIFICIAL TYMPANIC MEMBRANE | 29/09/2016 |
| | DEVICES AND USES | |
| WO2018117907 | SHAPE MEMORY POLYMER | 28/06/2018 |
| | COMPOSITE FOR 3D PRINTING OF | |
| | MEDICAL ITEMS | |
| WO2018218085 | THREE-DIMENSIONAL PRINTED | 29/11/2018 |
| | ORGANS, DEVICES, AND MATRICES | |
| WO2019178086 | ELECTROHYDRODYNAMIC | 19/09/2019 |
| | BIOPRINTER SYSTEM AND METHOD | |
| WO2020123945 | FABRIC MATERIAL FOR MEDICAL | 18/06/2020 |
| | DEVICES | |
| EP2363156 | METHOD OF FABRICATING | 07/09/2011 |
| | BIODEGRADABLE MEDICAL DEVICES | |
| | WITH ENHANCED MECHANICAL | |
| | STRENGTH AND PHARMACOLGOICAL | |
| | FUNCTIONS | |

### BRIEF SUMMARY OF THE INVENTION

One embodiment of the present invention includes a vascular implant, having a polymeric filter body and a central longitudinal axis, configured to be placed in a patient's blood conveying vessel portion.

The filter body preferably has a head and an outer periphery. The head can be circular, oval, rounded, polygonal or other shape that enables attachment of filter legs thereto.

Multiple filter legs preferably extend both radially and longitudinally from the head.

Each leg can preferably be tapering between the central longitudinal axis and the outer periphery.

Each leg preferably has a proximal end portion next to the head and a distal portion with an anchor for engaging a vessel wall.

Preferably, there is a weakened break point on one or more of the legs.

In one or more embodiments, the filter body and legs are 3D printed.

In one or more embodiments, the body and legs are 3D printed of a bioresorbable material.

In one or more embodiments, the head is positioned at the central longitudinal axis.

In one or more embodiments, each leg tapers. Preferably, the thickness of each leg is encompassed in a range of a minimum thickness of about 0.2 mm to a maximum thickness of about 4.0 mm.

In one or more embodiments, the weakened break point is an indentation.

In one or more embodiments, the weakened break point is a score.

In one or more embodiments, the polymeric body is too soft to be machined.

In one or more embodiments, the polymeric body has a durometer reading of between 35 Shore A and 75 Shore D.

In one or more embodiments, the head has a ring, annular shape, oval or rounded or polygonal shape.

In one or more embodiments, the filter body and legs define a vena cava filter.

In one or more embodiments, the filter body and legs define an occlusion device.

In one or more embodiments, a vascular implant has a filter body having a central longitudinal axis and configured to be placed in a patient's blood conveying vessel portion. The filter body has a head and an outer periphery. Multiple filter legs preferably extend both radially and longitudinally from the head. Each leg has a proximal end portion next to said head and a distal portion with anchor for engaging a vessel wall. There is a weakened break point on one or more of said legs.

In one or more embodiments, the filter body is of a material that is too soft to be machined.

In one or more embodiments, the head is in the form of a ring.

In one or more embodiments, the body and legs are 3D printed of a bioresorbable material, such as a bioresorbable polymer.

In one or more embodiments, the polymeric body is too soft to be machined, being of a material having a durometer reading below approximately 20 Shore D.

In one or more embodiments, at least some of said anchors are hook shaped.

The diameter of the present invention can vary depending on location as would be recognized and known to one of ordinary skill in the art. For inferior vena cava (IVC) pulmonary embolism prevention the diameter can be between about 15-30 mm. For iliofemoral pulmonary embolism prevention the diameter can be between about 6 and 20 mm. For other smaller vessels for occlusion, the diameter can be between about 2 and 8 mm. Lengths can be between about 5-50 mm.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is an elevation view of a preferred embodiment of the apparatus of the present invention;

FIG. 2 is a top view of a preferred embodiment of the apparatus of the present invention;

FIG. 3 is a fragmentary view of a preferred embodiment of the apparatus of the present invention; and

FIG. 4 is a fragmentary view of a preferred embodiment of the apparatus of the present invention.

### DETAILED DESCRIPTION OF THE INVENTION

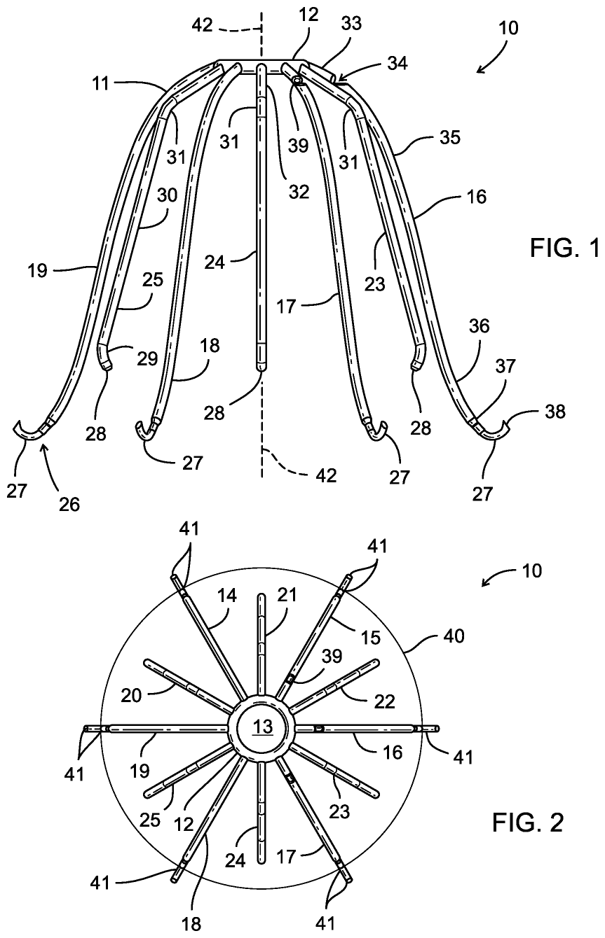

FIGS. 1-4 show a preferred embodiment of the apparatus of the present invention, designated generally by the numeral 10. Vascular implant 10 has a body 11 with central longitudinal axis 42. Body 11 can be polymeric and can be of a bioresorbable material such as a bioresorbable polymeric material. When of a bioresorbable material, the bioresorption starts as soon as the implant is exposed to the blood and like many of the standard polymers degrades over time via polymer breakdown and absorption of the byproducts. This bioresorption can be tuned as needed to ensure the implant maintains structural filtering integrity until transient risk of pulmonary embolism has subsided. Body 11 includes a head in the form of a ring, also referred to as an annular member 12 and has an outer periphery. In this example, the outer periphery lies in the longitudinal at a distance from the head, and the implant is to be placed in the vessel with the head upstream, in a direction of bloodflow, from the outer periphery. The body 11 is open at the bottom, and seen in radial direction, the bottom is wider than the head, more specifically in this embodiment the outer periphery is located at the bottom of the body 11. The body 11 has a frusto-conical shape with an open base, with the head 12 forming the top and the outer periphery the base of the conus. In this embodiment, the frusto-conical shape has a bell-shaped form, where at the top the conus is curved inwards, towards the central axis and at the bottom curved outwards.

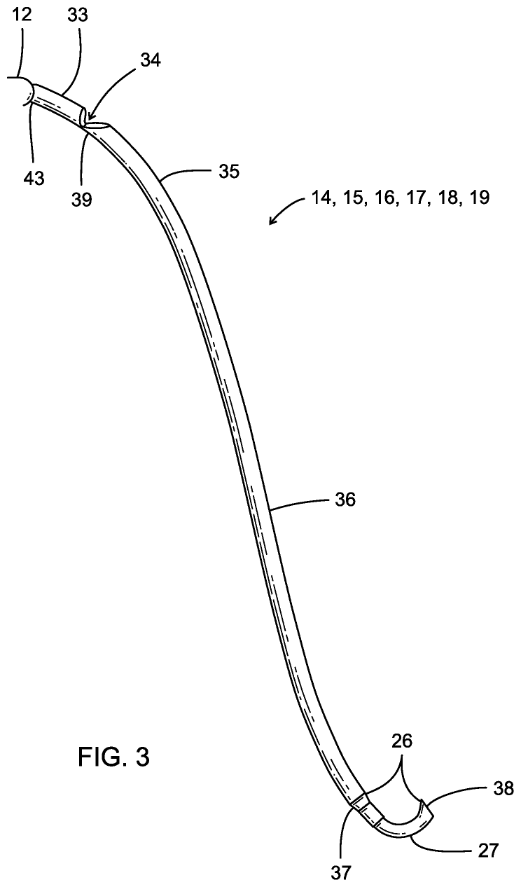

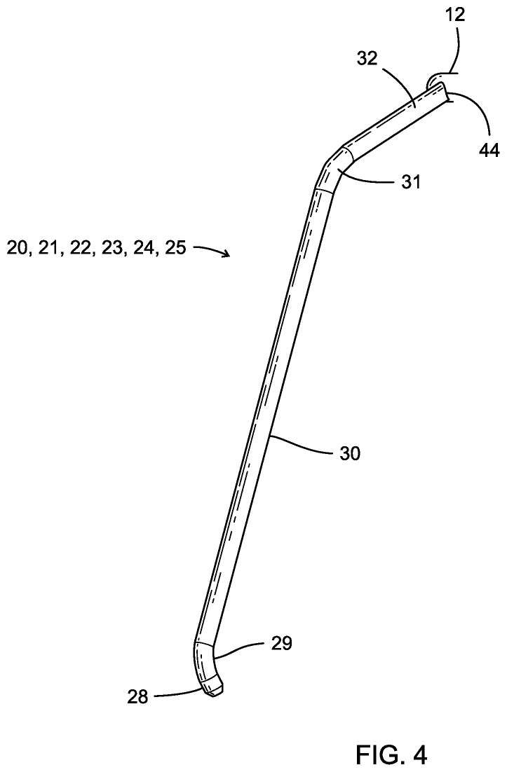

The body 11 comprises multiple appendages, also referred to as legs 14-19, which extend both radially and longitudinally from the head, towards the outer periphery, in this embodiment up to the outer periphery. The part of the appendages 14-19 which in radial direction is most distant from the longitudinal axis of the body 11 may form the outer periphery. Each of the legs 14-19 has a proximal end portion next to the head and a distal portion with an anchor for engaging a vessel wall. In the shown embodiment, appendages 14-19 are affixed at attachment 43 to ring 12 (see FIG. 3) (with appendages or legs 20-25 affixed to ring 12 with attachment 44) (see FIG. 4). In this embodiment, the distal portion forms a free-end of the respective appendage 14-19. The appendages may form an open cell filter or a closed cell filter, for instance.

When the vascular implant is placed in the vessel, blood will flow, substantially in the longitudinal direction of the body 11, in the direction from the outer periphery towards the head and pass through the body 11 to be filtered. The blood will flow through the space between the legs 14-25, as well as in this embodiment through the opening 13 of the ring 12, and blood clots that cannot pass through this space and, if present, the opening are blocked from passing through.

Hood, ring or annular member 12 can have central opening 13 (see FIG. 2). This can reduce the fluid resistivity of the filter body. In one or more embodiments, body 11 is manufactured (e.g., in one piece) with a 3D printer. Body 11 can be of a soft material that is too soft to be machined. A durometer reading of body 11 can be between about 35 Shore A and 75 Shore D.
The implant 10 may comprise mutually differing appendages. For example, the implant 10 may comprise two or more sets of appendages, where between the sets the appendages differ but in each set the appendages are similar. In the shown embodiment, for instance the implant 10 comprises appendages 14-19 with anchors and appendages 20-25 without anchors. Additionally, or alternatively, the sets may differ in other aspects. In the shown embodiment, for instance, appendages 14-19 (see FIG. 3) are a first set of appendages that can be longer than appendages 20-25 (see FIG. 1) of a second set. Appendages 14-19 each have a distal end or distal end portion 26 having an anchor 27 such as a hook shaped anchor 27. Anchor 27 can have a sharp tip 38 (see FIG. 3) for enabling penetration of anchor 27 into a patient's vascular tissue 40 (e.g., inferior vena cava or iliofemoral vein). In FIG. 2, reference numeral 41 represents the penetrating portion of anchor 27 that penetrates vascular tissue 40. The penetration allows to anchor the implant in the respective vessel and to hold the implant in position.

As illustrated in the shown embodiment, one or more of the appendages may be provided with a weakened break point, also referred to as a weakened portion 39. Due to the breakpoint, the appendage will break away from the body a certain period in time after having been placed in the vessel, without intervention of a medical practitioner. Thereby, the attachment of the body to the vessel via that appendage is released. The need for a retrieval procedure on the patient to retrieve the implant can be obviated thereby. For example, some or all of the appendages that anchor in the vessel may be provided with weakened break points and upon breaking of a respective breakpoint the filter may fall towards the opposite side of the vessel and to e.g. be resorbed in the vessel. In one or more embodiments, one or more of the appendages 14-19 has a recess, notch, or score 34 that is next to a weakened portion 39 (see FIG. 3). The weakened portion 39 is made of a material that breaks down over time, e.g. by dissolving or resorbing. The depth of the cut of recess, notch or score 34 determines the time to failure. In FIGS. 1 and 3, the notch is shown cut at the deep end of the range (though the notch need not be cut at the deep end of the range). Weakened portion 39 can be on an appendage that has an anchor. As weakened portion 39 dissolves or resorbs, the appendage portion distally of recess, notch, or score 34 will break away from the remainder of body 11. The appendage portion distally of recess, notch, or score 34 may then e.g. breakdown, e.g. dissolve or resorb into vascular tissue 40. The body 11 may be implemented such that all of body 11 will also resorb into the vascular tissue 40. When an appendage breaks at the weakened area 39 next to notch 34, it is a controlled failure. The leg would resorb into the wall/vascular tissue and the remaining portion of the filter would fall to the other/opposing side of the vessel and resorb into the wall/vascular tissue. This configuration provides a way to bias the filter to resorb where desired (desired location).

FIGS. 3-4 show appendages 14-19 (FIGS. 3) and 20-25 (FIG. 4) in more detail. Appendages 14-19 each have upper section 33, lower section 36 and middle section 35. Upper 33 and lower 36 sections can be curved (all or part of sections 33, 36 can be curved). Anchor 27 extends from or attaches to lower section 36 at tapered section 37 (see FIG. 3).

In FIG. 4, appendages 20-25 each have distal end 28. Each appendage 20-25 has a curved section or bend 29 that curves toward body 11 central longitudinal axis 42 (see FIG. 1). Thus, appendages 20-25 will not anchor into the vessel. Each appendage 20-25 can have straight section 30, curved section or bend 31, and straight section 32. Straight section 32 affixes to annular member, ring or head 12 at attachment 44. Appendages 20-25 can be shorter than appendages 14-19. For example, as seen in FIG. 2, appendages 20-25 can be, seen in radial direction, be inside the perimeter defined by the distal ends 27 of the appendages 14-19 and when the implant is placed not touch the vessel.

The implant 10 could be deployed with either a femoral or jugular approach. Such a deployment could employ a pusher or pusher apparatus/mechanism such as one specified in one or more of the patents listed in Table 1. An example is U.S. Pat. No. 8,518,072 naming Jonathan Miller as inventor and assigned to C.R. Bard, Inc. Implant 10 could also be a balloon-mounted implant that is then expanded with balloon dilation, as seen, for example, in balloon expandable stents.

The vascular implant can thus be characterized by comprising a filter body comprising a central longitudinal axis and configured to be placed in a patient's blood conveying vessel portion, said filter body comprising a head and an outer periphery. The body comprising multiple filter legs extending both radially and longitudinally from said head, each of said legs comprising a proximal end portion next to said head and a distal portion with anchor for engaging a vessel wall and a weakened break point on one or more of said legs. Without limitation, the implant may further be characterized by one or more of the following statements.

Statement 1. A vascular implant, comprising:

a) a polymeric filter body comprising a central longitudinal axis and configured to be placed in a patient's blood conveying vessel portion;

b) said filter body comprising a head and an outer periphery;

c) multiple filter legs extending both radially and longitudinally from said head;

d) each said leg tapering between said axis and said outer periphery;

e) each leg comprising a proximal end portion next to said head and a distal portion with an anchor for engaging a vessel wall; and

f) a weakened break point on one or more of said legs.

Statement 2. The vascular implant of statement 1 wherein said filter body and legs are 3D printed.

Statement 3. The vascular implant of statement 2 wherein the body and legs are 3D printed of a bioresorbable material.

Statement 4. The vascular implant of any of the preceding statements wherein the head is positioned at said central longitudinal axis.

Statement 5. The vascular implant of any of the preceding statements wherein each said leg tapers from a minimum thickness to a maximum thickness.

Statement 6. The vascular implant of any of the preceding statements wherein said weakened break point is an indentation.

Statement 7. The vascular implant of any of the preceding statements wherein said weakened break point is a score.

Statement 8. The vascular implant of any of the preceding statements wherein said polymeric body is too soft to be machined.

Statement 9. The vascular implant of statement 8 wherein the polymeric body has a durometer reading of between 35 Shore A and 75 Shore D.

Statement 10. The vascular implant of any of the preceding statements wherein said head is ring shaped.

Statement 11. The vascular implant of any of the preceding statements wherein the filter body and legs define a vena cava filter.

Statement 12. The vascular implant of any of the preceding statements wherein the filter body and legs define an occlusion device.

Statement 13. A vascular implant, comprising:

a) a filter body comprising a central longitudinal axis and configured to be placed in a patient's blood conveying vessel portion;

b) said filter body comprising a head and an outer periphery;

c) multiple filter legs extending both radially and longitudinally from said head;

d) each leg comprising a proximal end portion next to said head and a distal portion with anchor for engaging a vessel wall;

e) a weakened break point on one or more of said legs; and

f) wherein the filter body is of a material that is too soft to be machined.

Statement 14. The vascular implant of any of the preceding statements wherein the head is in the form of a ring.

Statement 15. The vascular implant of statement 13 or 14 wherein said filter body and legs are 3D printed.

Statement 16. The vascular implant of statement 15 wherein the body and legs are 3D printed of a bioresorbable material.

Statement The vascular implant of statement 16 wherein said body is too soft to be machined Statement 18. The vascular implant of any of the preceding statements wherein at least some of said anchors are hook shaped.

Statement 19. The vascular implant of any of statements 13-18 wherein at least some of said anchors are hook shaped.

Statement 20. A vascular implant, comprising:

a) a filter body comprising a central longitudinal axis and configured to be placed in a patient's blood conveying vessel portion;

b) said filter body comprising a head in the form of an annular member with a central opening and an outer periphery;

c) multiple filter legs extending both radially and longitudinally from said head;

d) each leg comprising a proximal end portion that connects said annular member and a distal end portion;

e) a weakened break point on one or more of said legs; and

f) wherein the filter body is of a material that is too soft to be machined.

The following is a list of parts and materials suitable for use in the present invention.

PARTS LIST

| Part Number | Description |
|---|---|
| 10 | vascular implant |
| 11 | body |
| 12 | annular member/ring/head |
| 13 | central opening |
| 14 | appendage/leg |
| 15 | appendage/leg |
| 16 | appendage/leg |
| 17 | appendage/leg |
| 18 | appendage/leg |
| 19 | appendage/leg |
| 20 | appendage/leg |
| 21 | appendage/leg |
| 22 | appendage/leg |
| 23 | appendage/leg |
| 24 | appendage/leg |
| 25 | appendage/leg |
| 26 | distal end portion |
| 27 | anchor/hook shaped distal end |
| 28 | distal end |
| 29 | curved section/bend |
| 30 | straight section |
| 31 | curved section/bend |
| 32 | straight section |
| 33 | upper section |
| 34 | recess/notch/score |
| 35 | middle section |
| 36 | lower section |
| 37 | tapered section |
| 38 | sharp tip |
| 39 | weakened portion |
| 40 | vascular tissue |
| 41 | penetrating portion |
| 42 | central longitudinal axis |
| 43 | attachment |
| 44 | attachment |

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims.

1. A vascular implant, comprising:
a) a polymeric filter body having a central longitudinal axis and configured to be placed in a patient's blood conveying vessel portion;
b) said filter body having a head and an outer periphery;
c) multiple filter legs extending both radially and longitudinally from said head;
d) each said leg tapering between said axis and said outer periphery;
e) each leg having a proximal end portion next to said head and a distal portion with an anchor for engaging a vessel wall; and
f) a weakened break point on one or more of said legs.

2. The vascular implant of claim 1 wherein said filter body and legs are 3D printed.

3. The vascular implant of claim 2 wherein the body and legs are 3D printed of a bioresorbable material.

4. The vascular implant of claim 1 wherein the head is positioned at said central longitudinal axis.

5. The vascular implant of claim 1 wherein each said leg tapers from a minimum thickness to a maximum thickness.

6. The vascular implant of claim 1 wherein said weakened break point is an indentation.

7. The vascular implant of claim 1 wherein said weakened break point is a score.

8. The vascular implant of claim 1 wherein said polymeric body is too soft to be machined.

9. The vascular implant of claim 8 wherein the polymeric body has a durometer reading of between 35 Shore A and 75 Shore D.

10. The vascular implant of claim 1 wherein said head is ring shaped.

11. The vascular implant of claim 1 wherein the filter body and legs define a vena cava filter.

12. The vascular implant of claim 1 wherein the filter body and legs define an occlusion device.

13. A vascular implant, comprising:
a) a filter body having a central longitudinal axis and configured to be placed in a patient's blood conveying vessel portion;
b) said filter body having a head and an outer periphery;
c) multiple filter legs extending both radially and longitudinally from said head;
d) each leg having a proximal end portion next to said head and a distal portion with anchor for engaging a vessel wall;
e) a weakened break point on one or more of said legs; and
f) wherein the filter body is of a material that is too soft to be machined.

14. The vascular implant of claim 1 wherein the head is in the form of a ring.

15. The vascular implant of claim 13 wherein said filter body and legs are 3D printed.

16. The vascular implant of claim 15 wherein the body and legs are 3D printed of a bioresorbable material.

17. The vascular implant of claim 16 wherein said body is too soft to be machined.

18. The vascular implant of claim 1 wherein at least some of said anchors are hook shaped.

19. The vascular implant of claim 13 wherein at least some of said anchors are hook shaped.

20. A vascular implant, comprising:
a) a filter body having a central longitudinal axis and configured to be placed in a patient's blood conveying vessel portion;
b) said filter body having a head in the form of an annular member with a central opening and an outer periphery;
c) multiple filter legs extending both radially and longitudinally from said head;
d) each leg having a proximal end portion that connects said annular member and a distal end portion;
e) a weakened break point on one or more of said legs; and
f) wherein the filter body is of a material that is too soft to be machined.

21. The inventions substantially as shown and/or described herein.
